(19) United States
(12) Patent Application Publication
(10) Pub. No.: US 2026/0248422 A1
(43) Pub. Date: Aug. 27, 2026

(54) PERSONAL ELECTRONIC DEVICE AND WEARABLE SYSTEM WITH DERMAL-CONTACT ELECTROCHEMICAL SENSING, INDIVIDUALIZED BASELINE ENROLLMENT, AND SECURE MULTIMODAL STATE INFERENCE

(71) Applicant: XGenesis, Grand Cayman (KY)

(72) Inventor: Todd Darling, Parkland, FL (US)

(73) Assignee: XGenesis, Grand Cayman (KY)

(21) Appl. No.: 19/651,358

(22) Filed: Apr. 17, 2026

Related U.S. Application Data
(63) Continuation-in-part of application No. 18/887,187, filed on Sep. 17, 2024.

(51) Int. Cl. A61B 5/1477; A61B 5/00; A61B 5/145

(52) U.S. Cl. CPC A61B 5/1477; A61B 5/14532; A61B 5/4845; A61B 5/7264; A61B 2560/0223

(57) ABSTRACT

A personal electronic device and wearable sensing system includes one or more dermal-contact electrochemical sensors integrated into one or more skin-contact surfaces of the electronic device, wearable, accessory, or interactive electronic system. The electrochemical sensors obtain measurements indicative of one or more analyte classes associated with a user. One or more additional sensors obtain non-electrochemical measurements associated with the user. A sensor-fusion module derives electrochemical and non-electrochemical features and generates a unified state estimate. One or more inference modules determine a user-state output based on the unified state estimate and a comparison of electrochemical-derived information, or features derived therefrom, to an individualized baseline reference profile associated with the user. In some embodiments, the analyte classes include endogenous physiological markers including glucose-related markers, exogenous exposure analytes, or both. In some embodiments, a credential derived from the individualized baseline reference profile is portable across multiple authorized devices within a trusted ecosystem.

### CROSS-REFERENCE TO RELATED APPLICATIONS

This application is a continuation-in-part of U.S. Patent Application No. 18/887,187, entitled “Impairment Recognition and Intervention System, Method and Apparatus,” filed September 17, 2024, the entire disclosure of which is incorporated herein by reference for all purposes permitted by law. This application is also related to US Patent No. 12,211,243 entitled “Multimodal diagnosis system, method and apparatus” granted on January 28, 2025 and to US Patent No. 12,039,764 entitled “Multimodal diagnosis system, method and apparatus” granted on July 16, 2024, the entire disclosures of which are incorporated herein by reference for all purposes permitted by law.

This application claims priority to U.S. Patent Application No. 18/887,187 as to all subject matter common therewith and claims the filing date of the present application as to subject matter first disclosed herein, including, without limitation, dermal-contact electrochemical sensing integrated into personal electronic devices, wearable devices, accessory devices, and other interactive electronic systems; individualized physiological baseline enrollment and baseline reference architectures; secure enclave or trusted execution comparison of individualized baseline information; multimodal fusion of electrochemical-derived features with optical, inertial, thermal, acoustic, imaging, touch, and contextual features; and state-inference architectures employing endogenous and exogenous analyte classes.

### FIELD

The present disclosure relates generally to personal electronic devices, wearable devices, accessory devices, and interactive electronic systems and, more particularly, to systems and methods that integrate dermal-contact electrochemical sensing with individualized baseline enrollment, secure state inference, and multimodal sensor fusion in order to determine physiological, cognitive, behavioral, compliance-relevant, authentication-relevant, readiness-related, or diagnostic-support user state.

### BACKGROUND

Personal electronic devices and wearable devices increasingly include multiple sensors and inference modalities intended to assess user state, health status, activity, compliance, readiness, authentication, or behavioral condition. Such devices may typically include individually or in limited combinations, without limitation, optical sensors, inertial sensors, microphones, cameras, temperature sensors, touch sensors, radios, chemical-sensing components, and other hardware or software components capable of generating information relevant to a user’s physiological or behavioral condition.

Existing state-determination approaches often rely on generalized population models, isolated sensor streams, or threshold-oriented inference that may be insufficiently individualized for high-confidence determination of consequential user state. Inter-user variation in skin properties, perspiration chemistry, metabolism, hydration, medication use, environmental exposure, contact behavior, and baseline physiology may reduce the reliability of population-average inference when applied to a specific user.

In addition, many existing device architectures do not robustly distinguish environmental contamination, incidental contact transfer, therapeutic background analytes, benign physiological variance, transient contact artifacts, or user-specific persistent baseline features from truly consequential user-state deviations. A signal may be detected, yet its meaning may remain ambiguous if the system cannot determine whether the signal is normal for that particular user, environmentally induced, transient, or indicative of a meaningful state change.

### SUMMARY

Briefly stated, the present continuation-in-part discloses a personal electronic device and wearable system incorporating dermal-contact electrochemical sensing, individualized baseline enrollment, and secure multimodal state inference. In some embodiments, one or more electrochemical sensing elements are integrated into a wearable band, wearable case, device housing, bezel, button, touchscreen border, rear device surface, ear-contact region, finger-contact region, wrist-contact region, facial-contact region of a head-mounted or mixed-reality device, or other dermal-contact interface configured to contact a user during ordinary device use.

In some embodiments, the electrochemical sensing elements are configured to obtain measurements indicative of one or more analyte classes associated with exogenous exposure, endogenous physiological state, metabolic condition, hydration, stress, fatigue, inflammation, glucose-related state, alcohol exposure, pharmacological exposure, or other monitored conditions. In certain embodiments, analyte classes may further include gustatory-domain chemical properties of substances at or near a robotic contact interface, including allergen-class markers, pharmacological compound presence, temperature-indicative signatures, or composition-relevant chemical markers associated with substances being handled, presented, or administered to a user.

In some embodiments, the device performs an individualized baseline enrollment process in which the user’s dermal-contact electrochemical profile, and optionally one or more corroborating modality profiles, are captured during a verified, designated, or otherwise accepted reference condition to establish a personal baseline reference profile for future comparisons. As used herein, a “designated reference condition” or “sober-state verification” refers to a selected baseline enrollment condition intended to represent a stable, accepted, or verified user state for later comparative inference.

In some embodiments, subsequent measurements are compared with the individualized baseline reference profile, optionally within a secure enclave, trusted execution environment, or functionally comparable hardware-isolated processing environment, thereby permitting privacy-preserving user-specific inference while reducing reliance on raw-data export or cloud-dependent comparison.

In some embodiments, electrochemical-derived features are fused with one or more additional modalities including, without limitation, optical sensing, photoplethysmography, inertial sensing, motion sensing, touch-derived features, acoustic sensing, temperature sensing, imaging, environmental sensing, and contextual device-usage information to determine whether a detected deviation reflects ordinary user variation, environmental contamination, a benign persistent baseline feature, or a consequential user-state change.

In some embodiments, the individualized baseline architecture is used not only for user-state inference but also for user authentication, longitudinal trend tracking, personalized model calibration, mitigation of user-specific sensing bias, adaptive alerting, diagnostic support, screening support, secure gating of device functions, transfer of a user’s baseline-derived credential across multiple authorized devices within a trusted ecosystem, or interaction with authorized robotic or other interactive electronic systems.

Dermal-contact electrochemical sensing provides a distinct measurement domain that may supplement or ground-truth other sensing modalities. At a skin-contact interface, one or more analytes, metabolites, or physiological markers may be detected or inferred through electrochemical interaction with perspiration, surface chemistry, or contact-mediated biosensing elements. However, standalone electrochemical sensing may still be insufficient if it is not individualized, securely handled, and interpreted together with corroborating modalities.

There therefore exists a need for a device architecture that combines dermal-contact electrochemical sensing with individualized baseline enrollment, multimodal corroboration, and secure on-device comparison so that a personal electronic device, wearable device, or related interactive electronic system can determine user state with greater specificity, privacy, adaptability, and resilience than isolated population-threshold sensing.

There further exists a need for a persistent user-specific baseline reference profile that permits later measurements to be interpreted relative to an enrolled personal reference state rather than exclusively by comparison to generalized norms. Such a baseline reference profile may be valuable in contexts including, without limitation, wellness monitoring, user authentication, compliance determination, readiness assessment, adaptive device behavior, longitudinal tracking, or safety-related function gating.

Such individualized architectures may also be useful in diagnostic-support and screening-support contexts, particularly where user-specific baseline deviation is more informative than comparison to generalized population norms.

There also exists a need for architectures in which individualized baseline profiles, feature comparisons, deviation determinations, and related user-state outputs are performed within a secure enclave, trusted execution environment, functionally comparable hardware-isolated processing domain, or other privacy-preserving edge-processing layer so that individualized inference can occur while reducing exposure of sensitive physiological information.

In further embodiments, the disclosed dermal-contact sensing, individualized baseline enrollment, and secure multimodal state-inference architectures may be implemented in other interactive electronic systems, including robotic platforms and humanoid systems configured for recurring dermal, facial, proximity, or multimodal interaction with human users.

### BRIEF DESCRIPTION OF THE DRAWINGS

FIG. 1 illustrates a personal electronic device and wearable sensing architecture including dermal-contact electrochemical sensing, multimodal sensor fusion, optional secure state inference, credential generation, and secure logging.

FIG. 2 illustrates example dermal-contact integration locations including wearable bands, device housings, wrist-contact regions, touch surfaces, rear surfaces, ear-contact structures, ring interiors, stylus grips, keyboard palm-contact areas, robotic interface surfaces, and facial-contact regions of head-mounted devices.

FIG. 3 illustrates an individualized baseline enrollment and physiological handshake workflow including designated reference condition verification (or sober-state verification), electrochemical and corroborating modality capture, feature extraction, baseline reference profile storage, portable baseline credential issuance, and adaptive baseline refresh and drift management.

FIG. 4 illustrates secure enclave or trusted execution processing of baseline comparison and state inference.

FIG. 5 illustrates multimodal corroboration logic distinguishing baseline-consistent state, environmental contamination, benign background-analyte condition, consequential state deviation, and security-relevant deviation, together with corresponding response logic for each classification.

FIG. 6 illustrates analyte-class embodiments including exogenous exposure analytes and endogenous physiological markers including glucose-related markers.

FIG. 7 illustrates cross-device credential portability in which an enrolled baseline-derived credential is used across multiple authorized devices within a trusted ecosystem.

### DETAILED DESCRIPTION

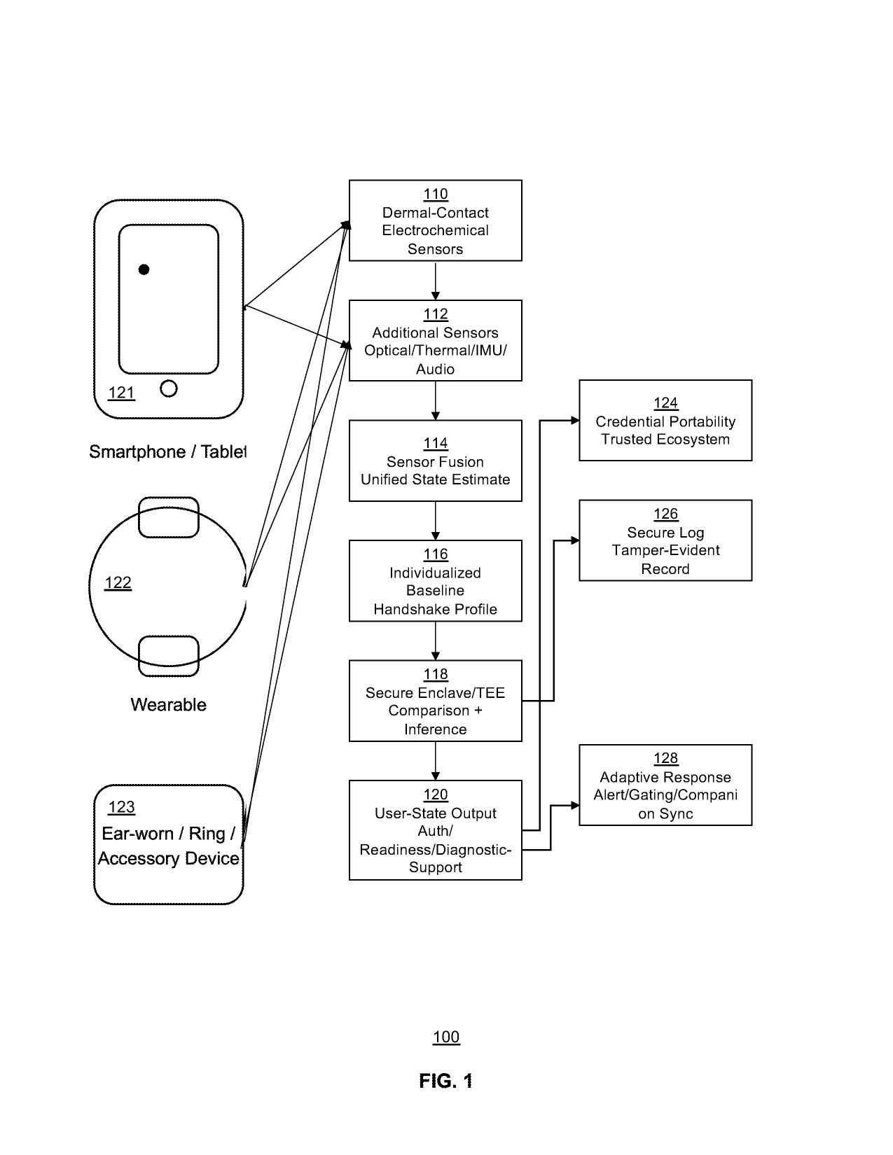

The present disclosure with reference to FIG. 1 extends the broader impairment recognition, intervention, and multimodal sensing concepts of the parent architecture 100 into personal electronic devices 121, wearable devices 122, accessory devices 123, and related interactive electronic systems. In contrast to vehicle-centered embodiments, the present disclosure focuses on dermal-contact electrochemical sensing integrated into personal electronics and wearables, combined with individualized baseline enrollment and secure state inference.

### Device Architecture

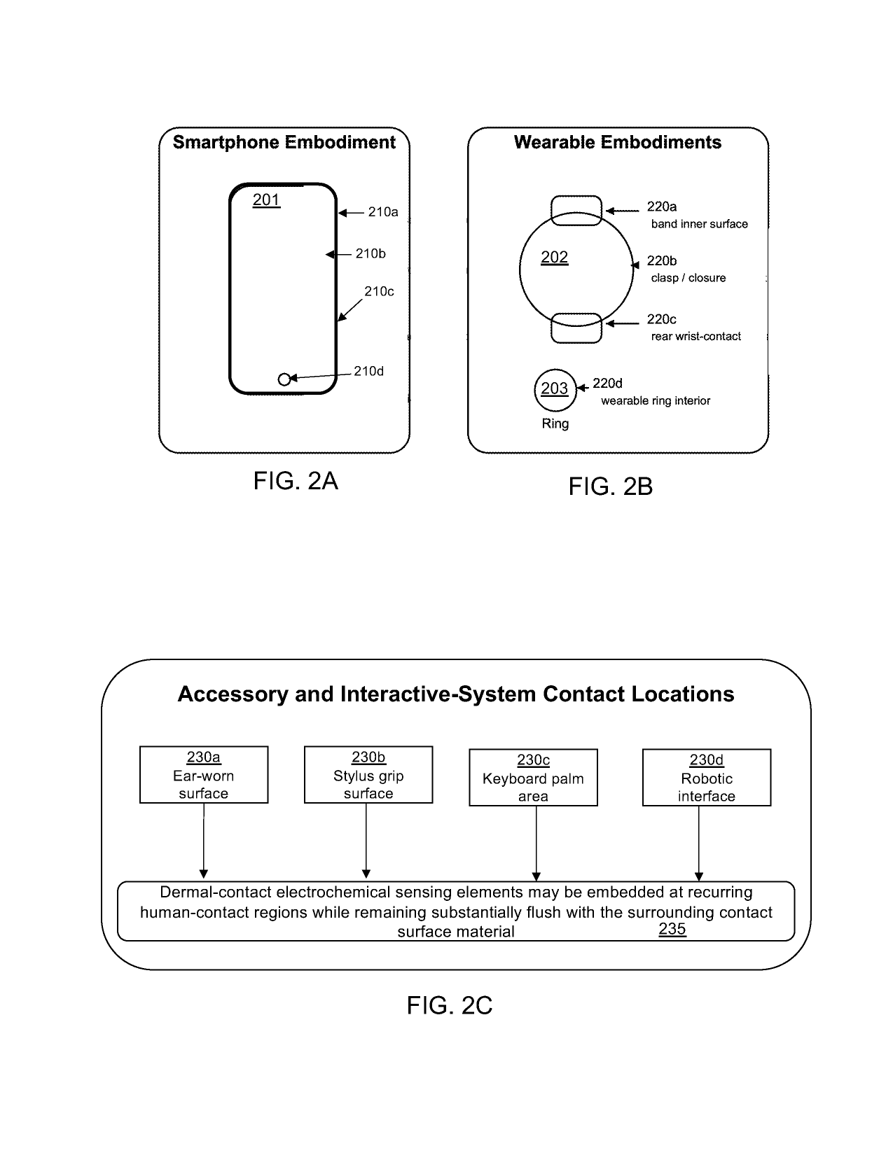

In certain embodiments with reference to FIGS. 1, 2A, 2B, and 2C, a device (121 or 201, 122 or 202 or 123 or 203) includes one or more dermal-contact electrochemical sensing elements 110 positioned at locations intended to contact the skin during ordinary use. Such locations may include, without limitation, a watch-band inner surface 220a, band clasp region 220b, wrist-contact field 220c, wearable ring interior 220d, earbud or headphone skin-contact surface, 230a smartphone side rail 210a, rear device surface 210c, button 210d, bezel-adjacent region 210b, touch border, stylus grip 230b, tablet edge, keyboard palm-contact area 230c, accessory-contact surface, or facial-contact region of a head-mounted or mixed-reality device.

Electrochemical sensing elements 235 or others may include one or more fuel-cell-based elements, enzyme-based biosensors, antibody-based biosensors, aptamer-based elements, ion-selective elements, impedance-based electrochemical elements, or functionally comparable contact-mediated sensing structures. The disclosure is not limited to any single electrochemical chemistry.

In wrist-worn embodiments, one or more sensing elements may be arranged to increase effective skin-contact area and maintain reliable signal acquisition during ordinary wear notwithstanding variable perspiration rates, motion, fit, or intermittent contact pressure. In certain implementations, the device may use repeated sampling, contact-quality estimation, averaging over time, or multimodal corroboration to accommodate lower perspiration density at wrist-contact regions relative to palmar contact surfaces.

In certain embodiments, one or more dermal-contact or proximity-contact electrochemical sensing elements may be integrated into contact surfaces of a robotic platform (230d), humanoid system, or other interactive electronic system configured for recurring physical interaction with human users. In such embodiments, the contact surface may include, without limitation, a robotic hand or fingertip surface, a robotic arm contact region, a food-handling or object-presentation interface, a caregiving-assist contact surface, or other surface through which the robotic system makes recurring contact with a human user or with objects presented to a human user. In certain implementations, one or more gustatory-domain sensing elements may be incorporated into or adjacent to such contact surfaces, configured to obtain electrochemical measurements indicative of one or more chemical properties of substances at or near the contact interface, including without limitation temperature, chemical composition, allergen-class markers, pharmacological compound presence, or other analyte classes relevant to the safety, suitability, or compliance-relevance of a substance being handled, presented, or administered to a human user. Such gustatory-domain electrochemical measurements may be fused with one or more additional modality inputs 112 including thermal sensing, inertial sensing, optical sensing, and tactile contact sensing within the sensor-fusion module 114 to produce a unified state estimate that is evaluated against an individualized baseline reference profile 116 associated with the user. In certain embodiments, this architecture supports safety-relevant determinations in contexts including, without limitation: elder care and assisted living environments, in which a robotic system may verify the temperature, composition, or medication content of food or liquid prior to presentation to a user whose enrolled profile reflects dietary restrictions, swallowing function parameters, or therapeutic medication requirements; clinical and hospital settings, in which a robotic system may verify the chemical identity or allergen status of a substance prior to administration, with the determination recorded in a tamper-evident log 126 providing an independently reviewable evidentiary record; school and institutional food service environments, in which a robotic system may detect allergen-class markers in food or drink prior to serving a user whose enrolled profile identifies life-threatening allergen sensitivities; and home robotic assistant contexts, in which a robotic system integrated with a user's enrolled individualized baseline reference profile may provide personalized dietary safety and health-relevant monitoring during food preparation, storage management, or meal service. In all such embodiments, no single gustatory-domain electrochemical detection event constitutes a standalone safety determination in the absence of corroboration from one or more additional modalities, and the governance determination is made relative to the individual user's enrolled baseline reference profile rather than against a generalized population threshold. Furthermore, in some embodiments, the baseline profile 116 can be stored in an enclave TEE (trusted environment) 118 that can also provide comparison and inferencing functions before placement in the secure log record 126. In some embodiments, the system can further provide a user-state output 120 while still preserving secure compartmentalization of sensitive data via a credential portability trusted ecosystem 124. The user-state output 120 can also operate in conjunction with an adaptive response alerting/gating/companion syncing function 128 as shown in FIG. 1.

### Analyte Classes

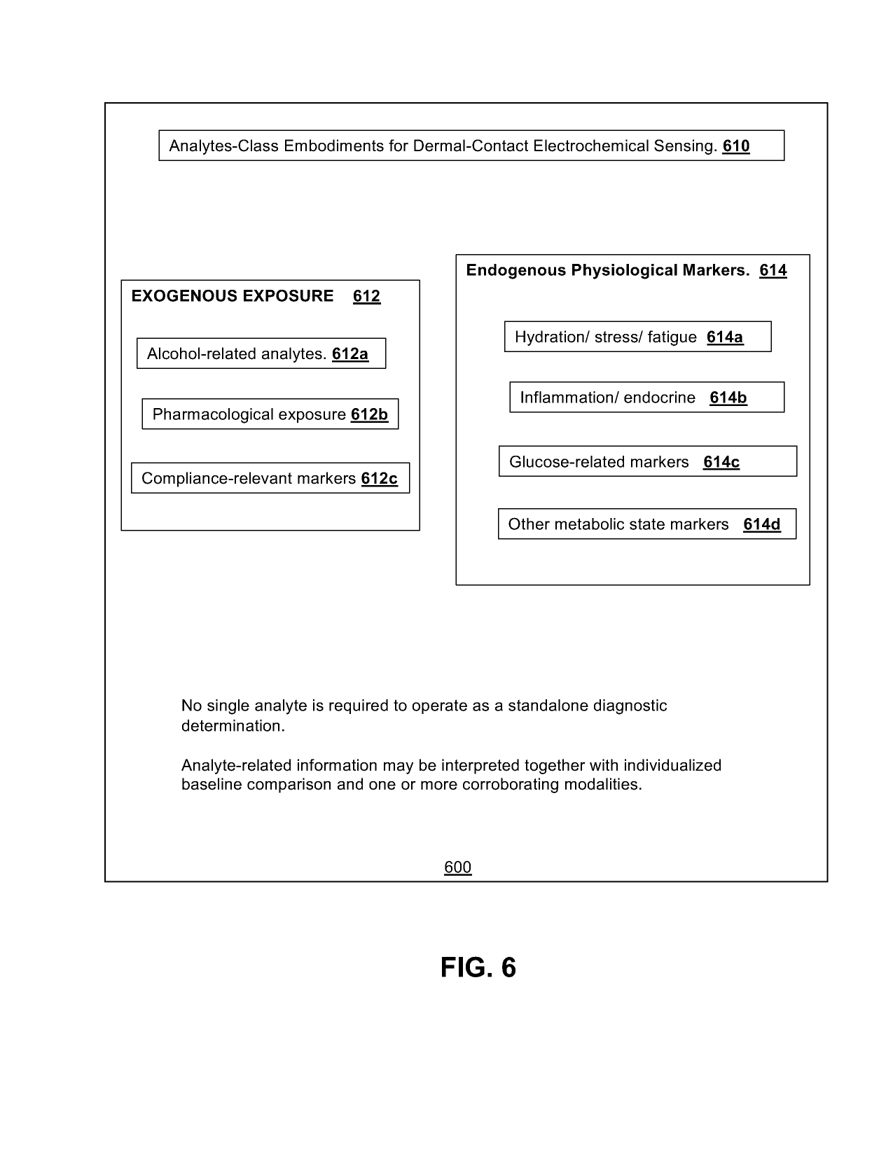

In some embodiments as illustrated by the system 600 of FIG. 6, monitored analyte classes 610 include one or more exogenous exposure analytes 612, including alcohol-related analytes 612a, pharmacological exposure analytes 612b, or compliance-relevant markers 612c. In further embodiments, monitored analyte classes include one or more endogenous physiological markers 614 associated with metabolic state, hydration, stress, fatigue (614a), inflammation, endocrine condition (614b), glucose-related state (614c), or other personal-health-relevant conditions (614d).

In some embodiments, glucose-related markers may be measured directly, indirectly, or inferentially through one or more electrochemical signatures associated with glucose state, glucose trend, or metabolic context, where technically validated. The disclosure is intended to encompass architectures in which glucose-related information is not interpreted in isolation but rather in combination with individualized baseline comparison and one or more corroborating modalities.

### Individualized Baseline Enrollment and Reference Profile

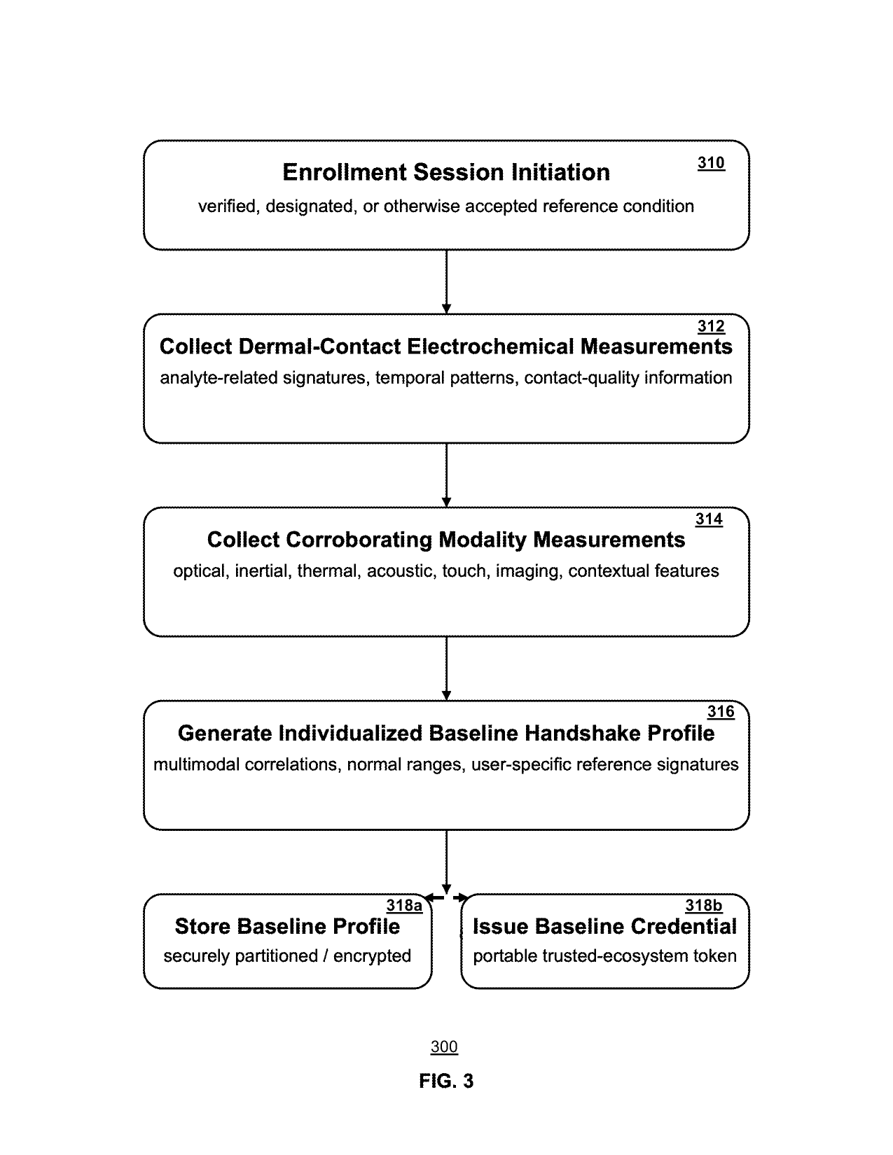

In certain embodiments, the device performs an enrollment procedure 300 as shown in FIG. 3 during which a personal baseline reference profile is established, stored (318a) and issued (318b). The enrollment may include an initiation at 310 and collection of one or more electrochemical measurements at 312 over time together with one or more additional modality measurements at 314 including optical, inertial, thermal, acoustic, touch, imaging, or contextual information. The resulting profile 316 may represent the user’s verified, designated, or otherwise accepted reference state.

The baseline reference profile 316 may include one or more analyte-related signatures, temporal-response patterns, contact-quality patterns, multimodal correlations, and user-specific normal ranges. The baseline may be stored locally, securely partitioned, cryptographically protected, or hardware-isolated.

In some embodiments, subsequent measurements are interpreted relative to the baseline reference profile rather than exclusively relative to generalized population-derived thresholds. This approach may reduce false inference arising from therapeutic background analytes, persistent physiological idiosyncrasies, medication use, optical variability, skin-contact variation, or user-specific metabolic characteristics.

As used herein, an “individualized baseline handshake profile” (116, 316) refers to an individualized baseline reference profile established during an enrollment event and subsequently used for comparative inference (118), credential generation (124), authentication, or coordinated state determination.

### Secure Enclave / Trusted Processing

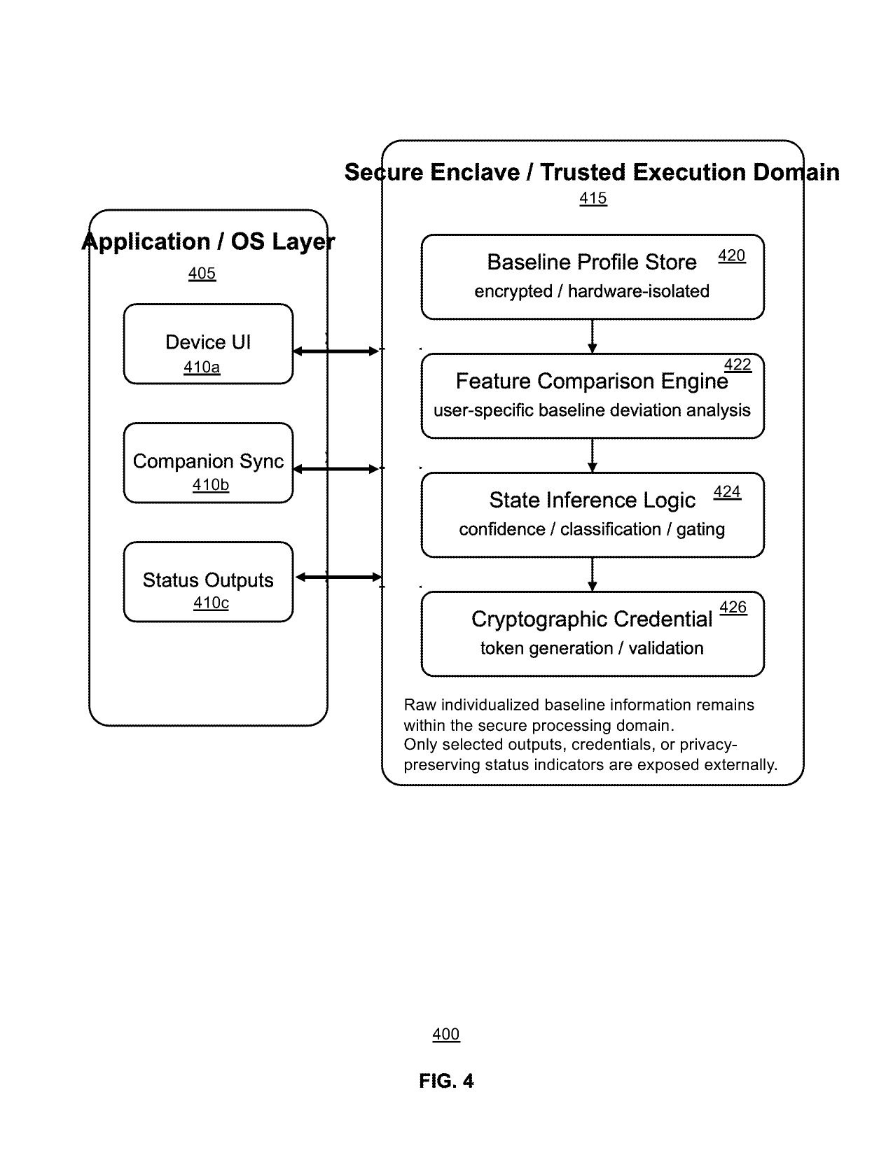

In certain embodiments as shown in the system 400 of FIG. 4, baseline storage 420, baseline comparison (422), feature generation (422), and state inference (424) are performed within a secure enclave, trusted execution environment, or functionally comparable hardware-isolated processing domain 415. Raw electrochemical data, or features derived therefrom, may therefore be processed on-device in a privacy-preserving manner.

Secure processing may permit the system to generate an output state, confidence score, deviation indicator, readiness determination, authentication result, or alert condition without exposing the full underlying individualized baseline profile to general application software, non-authorized device processes, or external services. An application or OS layer 405 can be used to securely access certain information such as status outputs 410c from the domain 415 using the device UI 410a and companion sync 410b. Again, raw individualized baseline information remains within the secure processing domain. Only selected outputs, credentials, or privacy-preserving status indicators are exposed externally using cryptographic credentials 426.

### Multimodal Corroboration

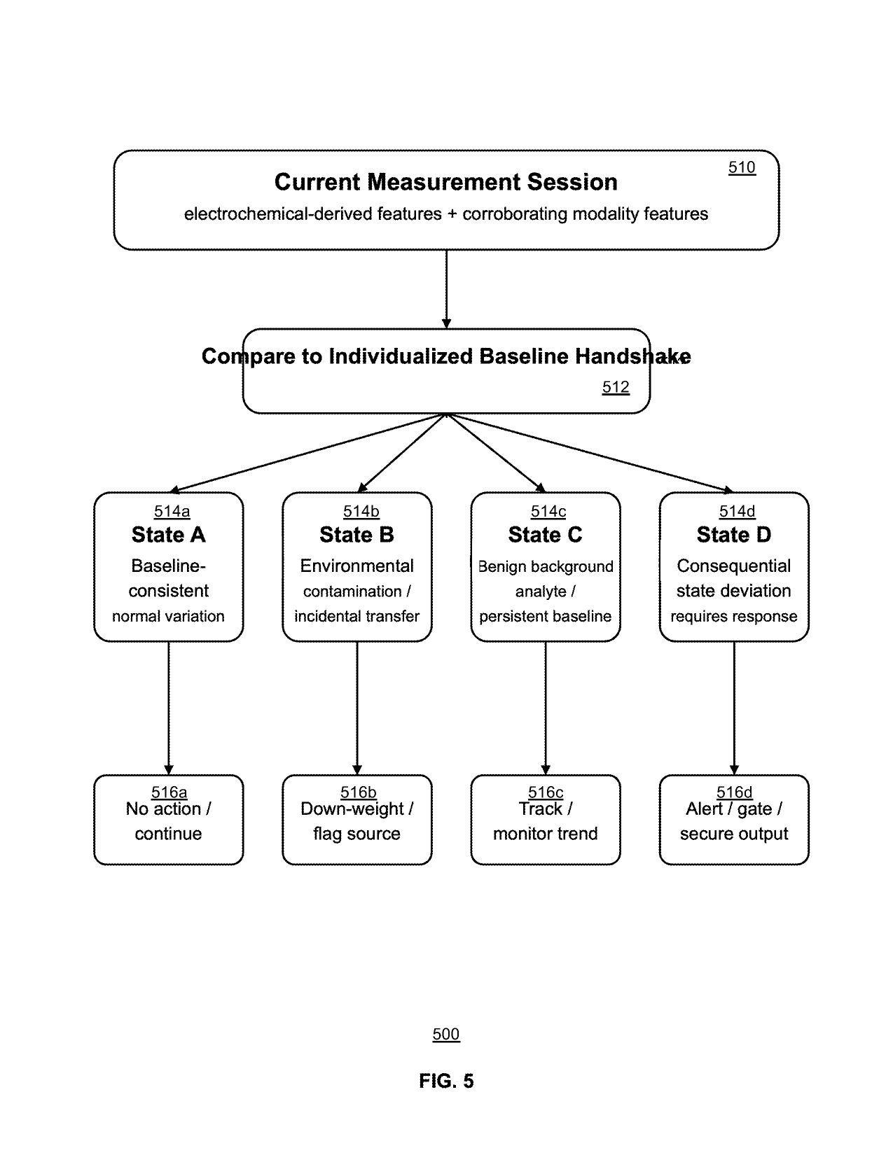

In certain embodiments, electrochemical-derived features are fused with one or more additional modality features as shown in the system 500 of FIG. 5, including, without limitation, photoplethysmography-derived features, optical absorbance or reflectance features, inertial features, thermal features, acoustic features, touch features, imaging features, skin-contact quality features, device-usage context, temporal patterns, and environmental measurements at 510.

Multimodal corroboration may be used to classify or compare a detected event into categories at 512 including, without limitation, baseline-consistent state 514a, environmental contamination 514b, benign background-analyte condition 514c, transient contact artifact, consequential physiological deviation 514d, compliance-relevant deviation, authentication-relevant deviation, or security-relevant deviation. As used herein, a “security-relevant deviation” may include a biosignature, analyte pattern, or multimodal state inconsistent with the enrolled user’s individualized baseline profile and indicative of potential unauthorized use, anomalous access, or other security-significant operation. Each state (514a, 514b, 514c, and 514d) can have a corresponding response or action such as no action/continue at 516a, or down-weight or flag source at 516b, or track or monitor trend at 516c, or alert, gate or secure output at 516d.

In some embodiments, the electrochemical sensing modality acts as a chemical calibration anchor, ground-truth signal, or user-specific reference modality for one or more additional sensing modalities. For example, a personalized optical-sensing model may be adjusted, validated, or refined using individualized electrochemical-derived information in order to improve calibration for that specific user.

In certain embodiments, the described sensing, baseline comparison, multimodal feature generation, and state-inference operations are performed substantially on-device at an edge-processing layer, thereby reducing reliance on remote computation and enabling privacy-preserving, low-latency operation. In further embodiments, one or more of such operations are performed within or in coordination with a secure enclave, trusted execution environment, or functionally comparable hardware-isolated processing domain.

### Functional Responses

In certain embodiments, a detected deviation from the individualized baseline reference profile may cause the device to perform one or more actions (516a-d) including display of an alert, adaptive sampling, invocation of a higher-confidence sensing mode, secure logging, gating of access to a protected function, transmission of a privacy-preserving status token, synchronization with an authorized companion device, or invocation of an authenticated robotic or interactive-electronic-system response.

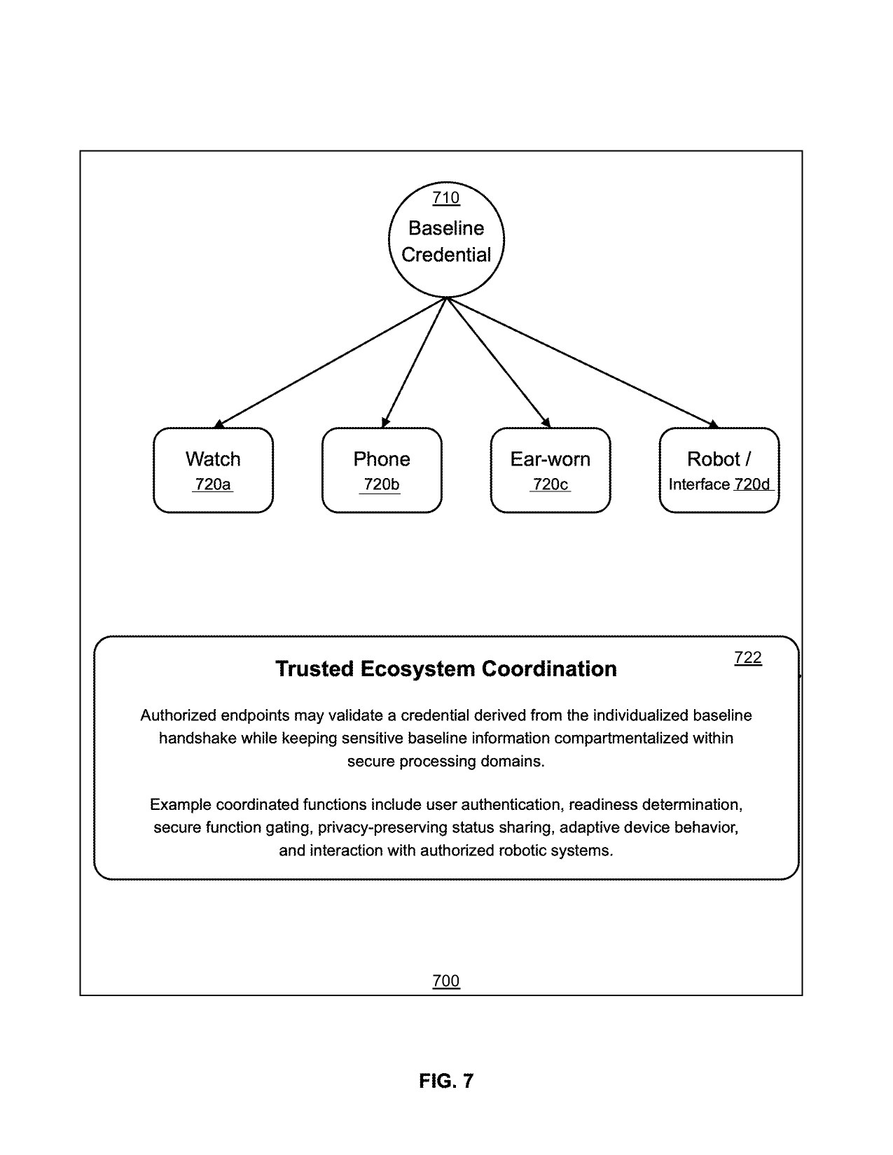

In some embodiments with reference to system 700 of FIG. 7, a user’s enrolled baseline reference profile, or a credential 710 derived therefrom, may be ported across multiple authorized devices within a trusted ecosystem such that a watch 720a, phone 720b, ear-worn device 720c, ring, headset, tablet, accessory device, robotic interface 720d, or other authenticated endpoint can participate in coordinated user-state determination at 722 while preserving secure compartmentalization of sensitive data.

In certain embodiments, the disclosed architecture may be used in diagnostic-support or screening-support workflows in which deviations from an individualized baseline reference profile are evaluated over time in conjunction with one or more corroborating modalities. In such embodiments, the system may generate a user-specific deviation indicator, trend output, or risk-related signal that supports further clinical, wellness, or device-mediated assessment without requiring that any single analyte or modality serve as a standalone diagnostic determination.

In certain embodiments, a credential derived from the individualized baseline reference profile may be represented as a cryptographically verifiable token (see 426 in FIG. 4) and may be stored, transmitted, or validated using a tamper-evident cryptographic record structure, including, without limitation, distributed ledger implementations, blockchain-based structures, append-only cryptographic logs, or other decentralized or distributed trust architectures.

In certain embodiments, the individualized baseline reference profile may be periodically refreshed, adaptively updated, or selectively re-enrolled in response to subsequently acquired measurements satisfying one or more stability, confidence, authorization, or drift-control criteria.

### Logging and Evidentiary Support

In certain embodiments, a secure log records one or more of enrollment events, baseline updates, comparison events, deviation classifications, confidence values, sensor-quality indicators, or device responses. The secure log may be cryptographically protected, hardware-isolated, tamper-evident, or implemented using a distributed or decentralized cryptographic record architecture.

Such logging may support auditing, clinical review, compliance review, safety review, model improvement, or user-controlled disclosure while maintaining privacy-preserving separation between raw individualized physiological data and externally disclosed summaries.

1. A personal electronic or wearable device system, comprising:
one or more dermal-contact electrochemical sensors integrated into one or more skin-contact surfaces of a personal electronic device, wearable device, accessory device, or interactive electronic system and configured to obtain electrochemical measurements indicative of one or more analyte classes associated with a user;
one or more additional sensors configured to obtain non-electrochemical measurements associated with the user;
a sensor-fusion module configured to derive electrochemical and non-electrochemical features and generate a unified state estimate; and
one or more inference modules configured to determine a user-state output based on the unified state estimate and a comparison of electrochemical-derived information, or features derived therefrom, to an individualized baseline reference profile associated with the user.

2. The system of claim 1, wherein the one or more skin-contact surfaces comprise at least one of a wearable-band surface, wearable ring interior, ear-contact surface, device housing surface, rear device surface, bezel-adjacent region, side rail, button surface, touch border, stylus grip, accessory-contact region, or facial-contact region of a head-mounted device, or a food-handling, object-presentation, or caregiving-assist contact surface of a robotic platform or humanoid system configured for recurring human interaction.

3. The system of claim 1, wherein the individualized baseline reference profile is established during an enrollment process that captures electrochemical measurements and one or more corroborating modality measurements while the user is in a designated reference condition.

4. The system of claim 1, wherein comparison to the individualized baseline reference profile is performed within a secure enclave, trusted execution environment, or functionally comparable hardware-isolated processing domain.

5. The system of claim 1, wherein the one or more analyte classes include at least one endogenous physiological marker.

6. The system of claim 5, wherein the at least one endogenous physiological marker includes one or more glucose-related markers.

7. The system of claim 1, wherein the one or more analyte classes include at least one exogenous exposure analyte associated with alcohol exposure or pharmacological exposure.

8. The system of claim 1, wherein the one or more additional sensors comprise one or more of optical sensors, photoplethysmography sensors, inertial sensors, thermal sensors, acoustic sensors, imaging sensors, touch sensors, environmental sensors, or contextual device-usage sensing modules.

9. The system of claim 1, wherein the sensor-fusion module is configured to classify a detected event as at least one of baseline-consistent state, environmental contamination, benign background-analyte condition, transient contact artifact, consequential physiological deviation, compliance-relevant deviation, authentication-relevant deviation, or security-relevant deviation.

10. The system of claim 1, wherein the electrochemical measurements are used as a calibration anchor for at least one additional sensing modality on a user-specific basis.

11. The system of claim 1, wherein the device is configured to generate a privacy-preserving status output without exposing the full individualized baseline reference profile outside a secure processing environment.

12. The system of claim 1, wherein a credential derived from the individualized baseline reference profile is portable across multiple authorized devices within a trusted ecosystem.

13. The system of claim 1, wherein the user-state output includes at least one of a diagnostic-support output, a screening-support output, or a longitudinal physiological trend output.

14. The system of claim 1, wherein at least one of sensing, baseline comparison, multimodal feature generation, or user-state inference is performed substantially on-device at an edge-processing layer.

15. The system of claim 12, wherein the credential derived from the individualized baseline reference profile is represented as a cryptographically verifiable token and is stored, transmitted, or validated using a tamper-evident cryptographic record structure.

16. The system of claim 12, wherein multiple authorized devices within a trusted ecosystem use the credential derived from the individualized baseline reference profile to participate in coordinated user-state determination while maintaining secure compartmentalization of individualized physiological information.

17. The system of claim 1, wherein access to a protected device function is gated based on a determination that current electrochemical-derived information, or features derived therefrom, sufficiently matches the individualized baseline reference profile.

18. A method comprising:
obtaining electrochemical measurements from one or more dermal-contact electrochemical sensors integrated into a personal electronic device, wearable device, accessory device, or interactive electronic system;
obtaining non-electrochemical measurements from one or more additional sensors;
comparing at least a portion of the electrochemical-derived information, or features derived therefrom, to an individualized baseline reference profile associated with a user;
generating a unified state estimate based on multimodal features; and
determining a user-state output based on the individualized baseline comparison and the unified state estimate.

19. The method of claim 18, further comprising establishing the individualized baseline reference profile during an enrollment event.

20. The method of claim 18, wherein the user-state output is associated with at least one of wellness, readiness, compliance, authentication, adaptive device behavior, secure function gating, or interaction with an authorized robotic or interactive electronic system.
